(19) United States
(12) Patent Application Publication
(10) Pub. No.: US 2007/0213540 A1
(43) Pub. Date: Sep. 13, 2007

(54) Process for the hydrogenation of imines

(75) Inventors: Juan Jose Almena Perea, Hanau (DE); Thomas Riermeier, Nidderau-Ostheim (DE); Axel Monsees, Frankfurt (DE); Renat Kadyrov, Frankfurt (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Degussa AG, Duesseldorf (DE)

(21) Appl. No.: 11/371,084

(22) Filed: Mar. 9, 2006

(51) Int. Cl. C07D 333/42; C07C 209/44

(52) U.S. Cl. 549/68; 564/415; 564/142

(57) ABSTRACT

The present invention relates to a process for the hydrogenation of an imines with hydrogen in the presence of iridium catalysts. In particular, the present invention relates to a process for the hydrogenation of imines with hydrogen under elevated pressure in the presence of an iridium catalyst and with or without an inert solvent, wherein the reaction mixture comprises a phosphonium chloride, bromide or iodide in the presence of an acid, which can be an organic or inorganic acid soluble or insoluble in the reaction mixture. Suitable imines are especially those that contain at least one 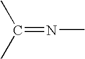 group. If the groups are substituted asymetrically and are thus compounds having a prochiral ketitine group, it is possible in the process according to the invention for mixtures of optical isomers or pure optical isomers to be formed if enantioselective or diastereoselective iridium catatalysts are used.

### BACKGROUND OF THE INVENTION

1. Field of the Invention

The present invention relates to a process for the hydrogenation of imines with hydrogen under elevated pressure in the presence of iridium catalysts and a halide, where in the reaction mixture optionally contains an acid.

2. Description of the Related Art

U.S. Pat. No. 4,994,615 describes a process for the asymmetric hydrogenation of prochiral N-arylketimines wherein iridium catalysts having chiral diphosphine ligands are used. U.S. Pat. No. 5,011,995 describes a process for the asymmetric hydrogenation of prochiral N-alkylketimines using the same catalysts. U.S. Pat. No. 5,112,999 discloses polynuclear iridium compounds and a complex salt of iridium, which contain diphosphine ligands, as catalysts for the hydrogenation of imines. U.S. Pat. No. 5,859,300 describes a process for the asymmetric hydrogenation of prochiral N-alkylketimines in the presence of an ammonium or metal halide and at least one solid acid with the exception of ion exchangers. U.S. Pat. No. 5,886,225 describes a process for the asymmetric hydrogenation of prochiral N-alkylketimines with an iridium catalyst in the presence of hydroiodic acid (HI). EP 0 691 949 B1 describes a process for the asymmetric hydrogenation of prochiral N-alkylketimines with an iridium catalyst in the presence of an ammonium or metal halide and an acid. WO 9521176 describes a process for the asymmetric hydrogenation of prochiral N-alkylketimines with an iridium (III) salt or a hydrate thereof, a diphosphine having secondary phosphine groups and an ammonium or metal halide.

Those homogeneous catalysis processes have proved valuable, although it is evident, especially in the case of relatively large batches or on an industrial scale, that the catalysts frequently tend to become deactivated to a greater or lesser extent depending on the catalyst precursor, the substrate and the diphosphine ligands that are used. In many cases, especially at elevated temperatures—for example at temperatures >25° C., which are necessary for a short reaction time—it is not possible to achieve complete conversion. For industrial applications of the hydrogenation process, therefore, the catalyst productivity is too low from the point of view of economic viability. To prevent the catalyst from being deactivated additives like ammonium salts, e.g. ammonium iodide, or acids are added to the reaction mixture.

R. Bedford et al. published in J. Organometallic Chem. 1997, 527(1-2), 75-82 the anomalous reactivity of triphenylarsine and triarylphosphines of low basicity with iridium complexes and the use of such complexes as precatalysts for imine hydrogenation. Oro, L. A. et al. published in Organometallics. 1999, 18(17), 3534-3546 the synthesis of labile hydrido complexes of iridium (III) by mixing iridium (I) dimers with phosphonium salt [HPⁱPr₃]BF₄ and their reactivity towards alkenes (insertion of alkenes in the Ir—H bond).

It has now been found, surprisingly, that a comparable catalyst activity can be retained or even increased when the reaction mixture essentially comprises a phosphonium halide instead of an ammonium halide and in some cases when the reaction mixture also contains an acid. Moreover, it has unexpectedly been found, although in the presence of an excess of a non chiral phosphine in the reaction mixture, that the ketimines can be reduced in an enantioselective way, even at reaction temperatures of more than 50° C. Additionally, in some cases the application of a phosphonium halide give better results than using an ammonium halide.

### SUMMARY OF THE INVENTION

Accordingly, it is the object of the present invention to provide a process for the hydrogenation of imines with hydrogen under elevated pressure in the presence of an iridium catalysts and with or without an inert solvent, wherein the reaction mixture comprises a phosphonium chloride, bromide or iodide in the presence or in the absence of an acid, which can be an organic or inorganic acid, soluble or insoluble in the reaction mixture.

### DETAILED DESCRIPTION OF THE PREFERRED EMBODIMENTS

In the process for the hydrogenation of imines in the present invention, suitable imines are especially those that contain at least one 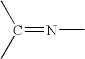
group. If the groups are substituted asymmetrically and are thus compounds having a prochiral ketimine group, it is possible in the process according to the invention for mixtures of optical isomers or pure optical isomers to be formed if enantioselective or diastereo-selective iridium catalysts are used. The imines may contain further chiral carbon atoms. The free bonds in the above formulae may be saturated with hydrogen or organic radicals having from 1 to 22 carbon atoms or organic hetero radicals having from 1 to 20 carbon atoms and at least one hetero atom from the group O, S, N and P. The nitrogen atom of the group 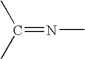
may also be saturated with NH₂ or a primary amino group having from 1 to 22 carbon atoms or a secondary amino group having from 2 to 40 carbon atoms. The organic radicals may be substituted, for example, by F, Cl, Br, C₁-C₄haloalkyl wherein halogen is preferably F or Cl, —CN, —NO₂, —CO₂H, —CONH₂, —SO₃H, —PO₃H₂, or C₁-C₁₂alkyl esters or amides, or by phenyl esters or benzyl esters of the groups —CO₂H, —SO₃H and —PO₃H₂. Adimine and ketimine groups are especially reactive, with the result that using the process according to the invention it is possible selectively to hydrogenate 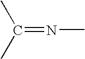
groups in addition to the —C═C— and/or C═O groups. Aldimine and ketimine groups are also to be understood to include 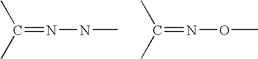
hydrazone and oxime groups.

The process according to the invention is suitable especially for the hydrogenation of aldimines, ketimines and hydrazones with the formation of corresponding amines and hydrazines, respectively. The ketimines are preferably N-substituted. It is preferable to use chiral iridium catalysts and to hydrogenate enantiomerically pure, chiral or prochiral ketimines to prepare optical isomers, the optical yields (enantiomeric excess, ee) being, for example, higher than 10%, preferably higher than 30%, and conversions of more than 80% being achievable. The optical yield indicates the ratio of the two stereoisomers formed, which ratio may be, for example, greater than 1.23:1 and preferably greater than 2:1.

The imines are preferably imines of formula I 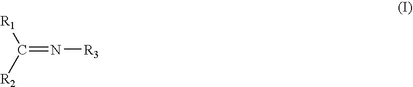
which are hydrogenated to form amines of formula II 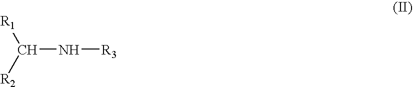
wherein

R₃ is preferably a substituent and wherein

R₃ is linear or branched C₁C₁₂alkyl, cycloalkyl having from 3 to 8 ring carbon atoms; heterocycloalkyl bonded via a carbon atom and having from 3 to 8 ring atoms and 1 or 2 hetero atoms from the group O, S and NR₆ a C₇C₁₆aralkyl bonded via an alkyl carbon atom or C₁-C₁₂alkyl substituted by the mentioned cycloalkyl or heterocycloalkyl or heteroaryl;

or wherein

R₃ is C₆-C₁₂aryl, or C₄-C₁₁heteroaryl bonded via a ring carbon atom and having 1 or 2 hetero atoms in the ring; R₃ being unsubstituted or substituted by —CN, —NO₂, F, Cl, C₁-C₁₂alkyl, C₁-C₁₂alkoxy, C₁-C₁₂alkylthio, C₁-C₆haloalkyl, —OH, C₆-C₁₂-aryl or -aryloxy or -arylthio, C₇-C₁₆-aralkyl or -aralkoxy or -aralkylthio, secondary amino having from 2 to 24 carbon atoms, —CONR₄R₅ or by —COOR₄, and the aryl radicals and the aryl groups in the aralkyl, aralkoxy and aralkylthio in turn being unsubstituted or substituted by —CN, —NO₂, F, Cl, C₁-C₄-alkyl, -alkoxy or -alkylthio, —OH, —CONR₄R₅ or by —COOR₄,

R₄ and R₅ are each independently of the other hydrogen, C₁-C₁₂alkyl, phenyl or benzyl, or

R₄ and R₅ together are tetra- or penta-methylene or 3-oxapentylene;

R₆ has independently the same meaning as given for R₄;

R₁ and R₂ are each independently of the other a hydrogen atom, C₁-C₁₂alkyl or cycloalkyl having from 3 to 8 ring carbon atoms, each of which is unsubstituted or substituted by —OH, C₁-C₁₂alkoxy, phenoxy, benzyloxy, secondary amino having from 2 to 24 carbon atoms, —CONR₄R₅ or by —COOR₄; C₆-C₁₂aryl or C₇-C₁₆aralkyl that is unsubstituted or substituted as R₃, or —CONR₄R₅ or —COOR₄, wherein R₄ and R₅ are as defined

hereinbefore; or

R₃ is as defined hereinbefore and R₁ and R₂ together are alkylene having from 2 to 5 carbon atoms that is optionally interrupted by 1 or 2 —O—, —S— or —NR₆— radicals, and/or unsubstituted or substituted by ═O or as R₁ and R₂ above in the meaning of alkyl, and/or condensed with benzene, pyridine, pyrimidine, fura, thiophene or pyrrole; or

R₂ is as defined hereinbefore and R₁ and R₃ together are alkylene having from 2 to 5 carbon atoms that is optionally interrupted by 1 or 2 —O—, —S— or —NR₆— radicals, and/or unsubstituted or substituted by ═O or as R₁ and R₂ above in the meaning of alkyl, and/or condensed with benzene, pyridine, pyrimidine, furan, thiophene or pyrrole.

The radicals R₁, R₂ and R₃ may contain one or more chirality centers.

R₁, R₂ and/or R₃ can be substituted in any desired positions by identical or different radicals, for example by from 1 to 5, preferably from 1 to 3, substituents.

Suitable substituents for R₁, R₂ and/or R₃ are: C₁-C₁₂-, preferably C₁-C₆-, and especially C₁-C₄-alkyl, -alkoxy or -alkylthio, e.g. methyl, ethyl, propyl, n-, iso- and tert-butyl, the isomers of pentyl, hexyl, octyl, nonyl, decyl, undecyl and dodecyl, and corresponding alkoxy and alkylthio radicals;

C₁-C₆-, preferably C₁-C₄-haloalkyl having preferably F and Cl as halogen, e.g. trifluoro- or trichloro-methyl, difluorochloromethyl, fluorodichloromethyl, 1,1-difluoroeth-1-yl, 1,1-dichloroeth-1-yl, 1,1,1-trichloro- or 1,1,1-trifluoroeth-2-yl, pentachloroethyl, penta-fluoroethyl, 1,1,1-trifluoro-2,2-dichloroethyl, n-perfluoropropyl, iso-perfluoropropyl, n-perfluorobutyl, fluoro- or chloro-methyl, difluoro- or dichloro-methyl, 1-fluoro- or 1-chloro-eth-2-yl or -eth-1-yl, 1-, 2- or 3-fluoro- or 1-, 2- or 3-chloro-prop-1-yl or -prop-2-yl or -prop-3-yl, 1-fluoro- or 1-chloro-but-1-yl, -but-2-yl, -but-3-yl or -but-4-yl, 2,3-dichloro-prop-1-yl, 1-chloro-2-fluoro-prop-3-yl, 2,3-dichlorobut-1-yl; C₆-C₁₂-aryl, -aryloxy or -arylthio, in which aryl is preferably naphthyl and especially phenyl, C₇-C₁₆-aralkyl, -aralkoxy and -aralkylthio, in which the aryl radical is preferably naphthyl and especially phenyl and the alkylene radical is linear or branched and contains from 1 to 10, preferably from 1 to 6 and especially from 1 to 3, carbon atoms, for example benzyl, naphthylmethyl, 1- or 2-phenyl-eth-1-yl or -eth-2-yl, 1-, 2- or 3-phenyl-prop-1-yl, -prop-2-yl or -prop-3-yl, with benzyl being especially preferred; the radicals containing the aryl groups mentioned above may in turn be mono- or poly-substituted, for example by C₁-C₄-alkyl, -alkoxy or -alkylthio, halogen, —OH, —CONR₄R₅ or by —COOR₅, wherein R₄ and R₅ are as defined; examples are methyl, ethyl, n- and iso-propyl, butyl, corresponding alkoxy and alkylthio radicals, F, Cl, Br, dimethyl-, methyl-ethyl- and diethyl-carbamoyl and methoxy-, ethoxy-, phenoxy- and benzyloxy-carbonyl;

halogen, preferably F and Cl;

secondary amino having from 2 to 24, preferably from 2 to 12 and especially from 2 to 6 carbon atoms, the secondary amino preferably containing 2 alkyl groups, for example dimethyl-, methylethyl-, diethyl-, methylpropyl-, methyl-n-butyl-, di-n-propyl-, di-n-butyl-, di-n-hexyl-amino;

—CONR₄R₅, wherein R₄ and R₅ are each independently of the other C₁-C₁₂—, preferably C₁-C₆-, and especially C₁-C₄-alkyl, or R₄ and R₅ together are tetra- or penta-methylene or 3-oxapentylene, the alkyl being linear or branched, e.g. dimethyl-, methylethyl-, diethyl-, methyl-n-propyl-, ethyl-n-propyl-, di-n-propyl-, methyl-n-butyl-, ethyl-n-butyl-, n-propyl-n-butyl- and di-n-butyl-carbamoyl;

—COOR₄, wherein R₄ is C₁-C₁₂-, preferably C₁-C₆-alkyl, which may be linear or branched, e.g. methyl, ethyl, n- and iso-propyl, n-, iso- and tert-butyl, and the isomers of pentyl, hexyl, heptyl, octyl, nonyl, decyl, undecyl and dodecyl.

R₁, R₂ or R₃ may contain especially functional groups, such as keto groups, —CN, —NO₂, carbon double bonds, N—O—, aromatic halogen groups and amide groups.

R₁, R₂ or R₃ as heteroaryl are preferably a 5- or 6-membered ring having 1 or 2 identical or different hetero atoms, especially O, S or N, which contains preferably 4 or 5 carbon atoms and can be condensed with benzene. Examples of heteroaromatics from which R₁ can be derived are furan, pyrrole, thiophene, pyridine, pyrimidine, indole and quinoline.

R₁, R₂ or R₃ as heteroaryl-substituted alkyl are derived preferably from a 5- or 6-membered ring having 1 or 2 identical or different hetero atoms, especially O, S or N, which contains preferably 4 or 5 carbon atoms and can be condensed with benzene. Examples of heteroaromatics are furan, pyrrole, thiophene, pyridine, pyrimidine, indole and quinoline.

R₁, R₂ or R₃ as heterocycloalkyl or as heterocycloalkyl-substituted alkyl contain preferably from 4 to 6 ring atoms and 1 or 2 identical or different hetero atoms from the group O, S and NR₆—, wherein R₆ is hydrogen, C₁-C₁₂alkyl, phenyl or benzyl. They can be condensed with benzene. It may be derived, for example, from pyrrolidine, tetrahydroform, tetrahydrothiophene, indane, pyrazolidine, oxazolidine, piperidine, piperazine or morpholine.

R₁, R₂ or R₃ as alkyl are preferably unsubstituted or substituted C₁-C₆-, especially

C₁-C₄-alkyl, which may be linear or branched. Examples are methyl, ethyl, iso- and n-propyl, iso-, n- and tert-butyl, the isomers of pentyl, hexyl, heptyl, octyl, nonyl, decyl, undecyl and dodecyl.

R₁, R₂ or R₃ as unsubstituted or substituted cycloalkyl contain preferably from 3 to 6, especially 5 or 6, ring carbon atoms. Examples are cyclopropyl, cyclobutyl, cyclopentyl, cyclohexyl, cycloheptyl and cyclooctyl.

R₁, R₂ or R₃ as aryl are preferably unsubstituted or substituted naphthyl and especially phenyl. R₁, R₂ or R₃ as aralkyl are preferably unsubstituted or substituted phenylalkyl having from 1 to 10, preferably from 1 to 6 and especially from 1 to 4 carbon atoms in the alkylene, the alkylene being linear or branched. Examples are especially benzyl, and 1-phenyleth-1-yl, 2-phenyleth-1-yl, 1-phenylprop-1-yl, 1-phenylprop-2-yl, 1-phenyl-prop-3-yl, 2-phenylprop-1-yl, 2-phenylprop-2-yl and 1-phenylbut-4-yl.

In R₂ and R₃ as —CONR₄R₅ and —COOR₄, R₄ and R₅ are preferably C₁-C₆, especially C₁-C₄-alkyl, or R₄ and R₅ together are tetramethylene, pentamethylene or 3-oxapentylene. Examples of alkyl are mentioned hereinbefore.

R₁ and R₂ together or R₁ and R₃ together as alkylene are preferably interrupted by 1 —O—, —S— or —NR₆—, preferably —O—. R₁ and R₂ together or R₁ and R₃ together form, with the carbon atom or with the —N═C group to which they are bonded, respectively, preferably a 5- or 6-membered ring. For the substituents the preferences mentioned hereinbefore apply. As condensed alkylene, R₁ and R₂ together or R₁ and R₃ together are preferably alkylene condensed with benzene or pyridine. Examples of alkylene are: ethylene, 1,2- or 1,3-propylene, 1,2-, 1,3- or 1,4-butylene, 1,5-pentylene and 1,6-hexylene. Examples of interrupted or ═O— substituted alkylene are 2-oxa-1,3-propylene, 2-oxa-1,4-butylene, 2-oxa- or 3-oxa-1,5-pentylene, 3-thia-1,5-pentylene, 2-thia-1,4-butylene, 2-thia-1,3-propylene, 2-methylimino-1,3propylene, 2-ethylimino-1,4-butylene, 2- or 3-methyl-imino-1,5-pentylene, 1-oxo-2-oxa-1,3-propylene, 1-oxo-2-oxa-1,4-butylene, 2-oxo-3-oxa-1,4-butylene, 1-oxa-2-oxo-1,5-pentylene.

Examples of Condensed alkylene are:

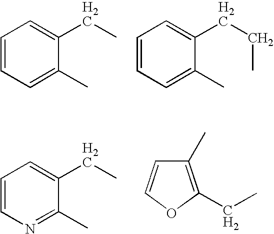

Examples of condensed and interrupted and unsubstituted or ═O-substituted alkylene are:

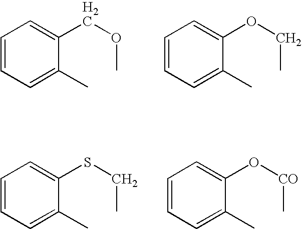

R₄ and R₅ are preferably each independently of the other hydrogen, C₁-C₄alkyl, phenyl or benzyl. R₆ is preferably hydrogen or C₁-C₄alkyl.

A further preferred group is formed by prochiral imines in which in formula I R₁, R₂ and R₃ are each different from the others and are not hydrogen.

In an especially preferred group, in formula I R₃ is 2,6-di-C₁-C₄alkylphen-1-yl and especially 2,6-dimethylphen-1-yl or 2-methyl-6-ethylphen-1-yl, R₁ is C₁-C₄alkyl and especially ethyl or methyl, and R₂ is C₁-C₄alkyl, C₁-C₄alkoxymethyl or C₁-C₄alkoxyethyl, and especially methoxymethyl.

Of those compounds, imines of formula 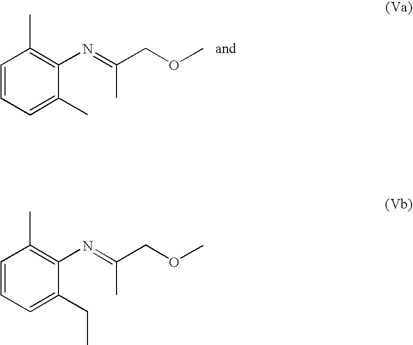
are especially important, as is the imine of the formula

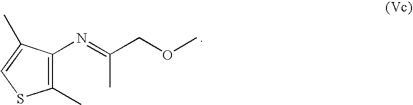

Imines of formula I are known or they can be prepared in accordance with known processes from aldehydes or ketones and primary amines.

The iridium catalysts are preferably homogeneous catalysts that are substantially soluble in the reaction medium. The term “catalyst” also includes catalyst precursors that are converted into an active catalyst species at the beginning of a hydrogenation. The catalysts preferably correspond to the formula III, IIIa, IIIb, IIIc and IIId,
[XIrYZ] (III),
[XIrY]⁺A⁻ (IIIa),
[YIrZ₄]⁻M⁺ (IIIb),
[YIrHZ₂]₂ (IIIc),
[YIrZ₃]₂ (IIId),
wherein X is two olefin ligands or a diene ligand, Y is a ditertiary diphosphine

(a) the phosphine groups of which are bonded to different carbon atoms of a carbon chain having from 2 to 4 carbon atoms, or

(b) the phosphine groups of which are either bonded directly or via a bridge group —CRₐR_{b}— in the ortho positions of a cyclopentadienyl ring or are each bonded to a cyclopentadienyl ring of a ferrocenyl, or

(c) one phosphine group of which is bonded to a carbon chain having 2 or 3 carbon atoms and the other phosphine group of which is bonded to an oxygen atom or a nitrogen atom bonded terminally to that carbon chain, or

(d) the phosphine groups of which are bonded to the two oxygen atoms or nitrogen atoms bonded terminally to a C₂-carbon chain;

with the result that in the cases of (a), (b), (c) and (d) a 5-, 6-, 7-, 8- or 9-membered ring is formed together with the Ir atom, the radicals Z are each independently of the other(s) Cl, Br or I, A⁻ is the anion of an oxy or complex acid, and M⁺ is a phosphonium cation, and Rₐ and R_{b}, are each independently of the other hydrogen, C₁-C₈alkyl, C₁-C₄fluoroalkyl, phenyl or benzyl or are phenyl or benzyl having from 1 to 3 C₁-C₄alkyl or C₁-C₄alkoxy substituents. R_{b} is preferably hydrogen. Rₐ is preferably C₁-C₄alkyl and especially methyl.

The diphosphine Y contains preferably at least one chiral carbon atom and is especially an optically pure stereoisomer (enantiomer or diastereoisomer), or a pair of diastereoisomers, since the use of catalysts containing those ligands leads to optical induction in asymmetric hydrogenation reactions.

X as an olefin ligand may be a branched or, preferably, linear C₂-C₁₂alkylene, especially C₂-C₆alkylene. Some examples are dodecylene, decylene, octylene, 1-, 2- or 3-hexene, 1-, 2- or 3-pentene, 1- or 2-butene, propene and ethene. X as a diene ligand may be open-chain or cyclic dienes having from 4 to 12, preferably from 5 to 8, carbon atoms, the diene groups preferably being separated by one or two saturated carbon atoms. Some examples are butadiene, pentadiene, hexadiene, heptadiene, octadiene, decadiene, dodecadiene, cyclopentadiene, cyclohexadiene, cycloheptadiene, cyclooctadiene and bridged cyclo-dienes such as norbomadiene and bicyclo-2,2,2-octadiene. Hexadiene, cyclooctadiene and norbomadiene are preferred.

The phosphine groups contain preferably two identical or different, preferably identical, unsubstituted or substituted hydrocarbon radicals having from 1 to 20, especially from 1 to 12 carbon atoms. Preference is given to diphosphines wherein the secondary phosphine groups contain two identical or different radicals from the following group: linear or branched C₁-C₁₂alkyl; unsubstituted or C₁-C₆alkyl- or C₁-C₆alkoxy-substituted C₅-C₁₂-cycloalkyl, C₅-C₁₂cycloalkyl-CH₂—, phenyl or benzyl; and phenyl or benzyl substituted by halogen (e.g. F, Cl or Br), C₁-C₆haloalkyl, (C₁-C₁₂alkyl)₃Si, (C₆H₅)₃Si, C₁-C₆haloalkoxy (e.g. trifluoromethoxy), —NH₂, phenyl₂N—, benzyl₂N—, morpholinyl, piperidinyl, pyrrolidinyl, (C₁-C₁₂alkyl)₂N—, -ammonium-X₁⁻, —SO₃M₁, —CO₂M₁, —PO₃M₁ or by —COO-C₁-C₆-alkyl (e.g. —COOCH₃), wherein M₁ is an alkali metal or hydrogen and X₁⁻ is the anion of a monobasic acid. M₁ is preferably H, Li, Na or K. A₁³¹ , as the anion of a monobasic acid, is preferably Cl⁻, Br⁻ or the anion of a carboxylic acid, for example formate, acetate, trichloroacetate or trifluoroacetate.

A secondary phosphine group may also be a radical of the formula 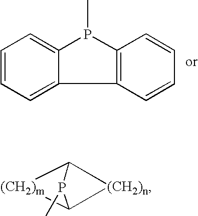
wherein

m and n are each independently of the other an integer from 1 to 10, and the sum of m+n is from 1 to 12, especially from 4 to 8. Examples thereof are [3.3.1]- and [4.2.1]-phobyl of the formulae

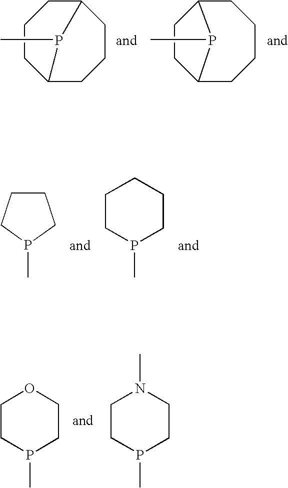

Examples of alkyl that preferably contains from 1 to 6 carbon atoms are methyl, ethyl, n-propyl, isopropyl, n-, iso- and tert-butyl and the isomers of pentyl and hexyl. Examples of unsubstituted or alkyl-substituted cycloalkyl are cyclopentyl, cyclohexyl, methyl- or ethyl-cyclohexyl and dimethylcyclohexyl. Examples of alkyl-, alkoxy- or haloalkoxy-substituted phenyl and benzyl are methylphenyl, dimethylphenyl, trimethylphenyl, ethyl-phenyl, methylbenzyl, methoxyphenyl, dimethoxyphenyl, trifluoromethylphenyl, bis-tri-fluoromethylphenyl, iris-trifluoromethylphenyl, trifluoromethoxyphenyl and bis-trifluoro-methoxyphenyl. Preferred phosphine groups are those that contain identical or different, preferably identical, radicals from the group C₁-C₆alkyl; cyclopentyl and cyclohexyl that are unsubstituted or have from 1 to 3 C₁-C₄alkyl or C₁-C₄alkoxy substituents, benzyl and, especially, phenyl that is unsubstituted or has from 1 to 3 C₁-C₄alkyl, C₁-C₄alkoxy, F, Cl, C₁-C₄fluoroalkyl or C₁-C₄fluoroalkoxy substituents.

Y as a diphosphine is preferably of formula IV, IVa, IVb, IVc or IVd,
R₇R₈P—R₉PR₁₀R₁₁ (IV),
R₇R₈P—O—R₁₂—PR₁₀R₁₁ (IVa),
R₇R₈P—NR_{c}—R₁₂—PR₁₀R₁₁ (IVb),
R₇R₈P—O—R₁₃—O—PR₁₀R₁₁ (IVc),
R₇R₈P—NR_{c}—R₁₃—NR_{c}—PR₁₀R₁₁ (IVd),
R₇R₈P—NR_{c}—R₉—PR₁₀R₁₁ (IVe)
wherein

R₇, R_{8,} R₁₀ and R₁₁ are each independently of the others a hydrocarbon radical having from 1 to 20 carbon atoms that is unsubstituted or substituted by C₁-C₆alkyl, C₁-C₆alkoxy, halogen, C₁-C₆haloalkyl, (C₁-C₁₂alkyl)₃Si, (C₆H₅)₃Si, C₁-C₆haloalkoxy, —NH₂, phenyl₂N—, benzyl₂N—, morpholinyl, piperidinyl, pyrrolidinyl, (C₁-C₁₂alkyl)₂N—, -ammonium-X₁⁻, —SO₃M₁, —CO₂M₁, —PO₃M₁ or by —COO—C₁-C₆-alkyl (e.g. —COOCH₃), wherein M₁ is an alkali metal or hydrogen and X₁⁻ is the anion of a monobasic acid;

R₉ is linear C₂-C₄alkylene that is unsubstituted or substituted by C₁-C₆alkyl, C₅-or C₆-cycloalkyl 6-cycloalkyl, phenyl, naphthyl or by benzyl; 1,2- or 1,3-cycloalkylene or -cycloalkenylene, -bicycloalkylene or -bicycloalkenylene having from 4 to 10 carbon atoms, each of which is unsubstituted or substituted by C₁-C₆alkyl, phenyl or by benzyl; 1,2- or 1,3-cycloalkylene or -cycloalkenylene, -bicycloalkylene or -bicycloalkenylene having from 4 to 10 carbon atoms, each of which is unsubstituted or substituted by C₁-C₆alkyl, phenyl or by benzyl, and in the 1- and/or 2-positions or in the 3-position of which methyl-ene or C₂-C₄alkylidene is bonded; 1,4-butylene substituted in the 2,3-positions by 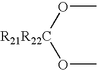
and unsubstituted or substituted in the 1,4-positions by C₁-C₆alkyl, phenyl or by benzyl, wherein R₂₁ and R₂₂ are each independently of the other hydrogen, C₁-C₆alkyl, phenyl or benzyl; 3,4- or 2,4-pyrrolidinylene or 2-methylene-pyrrolidin-4-yl the nitrogen atom of which is substituted by hydrogen, C₁-C₁₂alkyl, phenyl, benzyl, C₁-C₁₂alkoxycarbonyl, C₁-C₈acyl or by C₁-C₁₂alkylaminocarbonyl; or 1,2-phenylene, 2-benzylene, 1,2-xylylene, 1,8-naphthylene, 2,2′-dinaphthylene or 2,2′-diphenylene, each of which is unsubstituted or substituted by C₁-C₄alkyl;
or R₉ is a radical of the formula 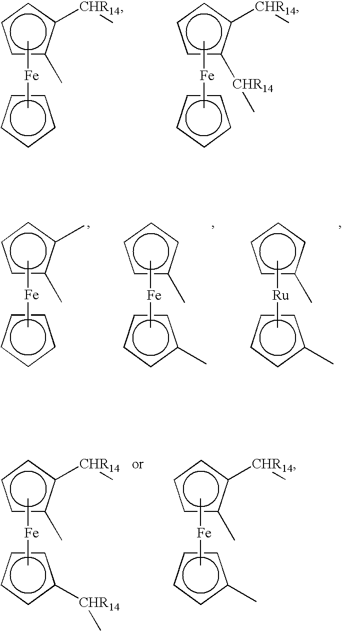
wherein R₁₄ is hydrogen, C₁-C₈alkyl, C₁-C₄fluoroalkyl, phenyl or phenyl having from 1 to 3 C₁-C₄alkyl or C₁-C₄alkoxy substituents;

R₁₂ is linear C₂— or C₃-alkylene that is unsubstituted or substituted by C₁-C₆alkyl, C₅-or C₆-cycloalkyl, phenyl, naphthyl or by benzyl; 1,2- or 1,3-cycloalkylene or -cycloalkenylene, -bicycloalkylene or -bicycloalkenylene having from 4 to 10 carbon atoms, each of which is unsubstituted or substituted by C₁-C₆alkyl, phenyl or by benzyl; or 1,2- or 1,3-cycloalkylene or -cycloalkenylene, -bicycloalkylene or -bicycloalkenylene, -bicycloalkylene or -bicycloalkenylene having from 4 to 10 carbon atoms, each of which is unsubstituted or substituted by C₁-C₆alkyl, phenyl or by benzyl, and in the 1- and/or 2-positions or in the 3-position of which methylene or C₂-C₄alkylidene is bonded; 3,4- or 2,4-pyrrolidinylene or 3-methylene-pyrrolidin-4-yl the nitrogen atom of which is substituted by hydrogen, C₁-C₁₂alkyl, phenyl, benzyl, C₁-C₁₂alkoxycarbonyl, C₁-C₈acyl or by C₁-C₁₂alkylaminocarbonyl; or 1,2-phenylene, 2-benzylene, 1,2-, 2,3- or 1,8-naphthylene, each of which is unsubstituted or substituted by C₁-C₄alkyl; and

R₁₃ is linear C₂alkylene that is unsubstituted or substituted by C₁-C₆alkyl, C₅- or C₆-cycloalkyl, phenyl, naphthyl or by benzyl; 1,2-cycloalkylene or -cycloalkenylene, -bicycloalkylene or -bicycloalkenylene having from 4 to 10 carbon atoms, each of which is unsubstituted or substituted by C₁-C₆alkyl, phenyl or by benzyl; 3,4-pyrrolidinylene the nitrogen atom of which is substituted by hydrogen, C₁-C₁₂alkyl, phenyl, benzyl, C₁-C₁₂alkoxycarbonyl, C₁-C₈acyl or by C₁-C₁₂alkylaminocarbonyl; or 1,2-phenylene that is unsubstituted or substituted by C₁-C₄alkyl, or is a radical, less two hydroxy groups in the ortho positions, of a mono- or di-saccharide, and

R_{c} is hydrogen, C₁-C₄alkyl, phenyl or benzyl.

R₇, R₈, R₁₀ and R₁₁ are preferably identical or different, preferably identical, radicals from the following group: C₁-C₆alkyl; cyclopentyl and cyclohexyl that are unsubstituted or have from 1 to 3 C₁-C₄alkyl or C₁-C₄alkoxy substituents, benzyl and, especially, phenyl that is unsubstituted or has from 1 to 3 C₁-C₄alkyl, C₁-C₄alkoxy, F, Cl, C₁-C₄fluoroalkyl or C₁-C₄fluoroalkoxy substituents.

A preferred subgroup of diphosphines Y is formed by those of the formulae 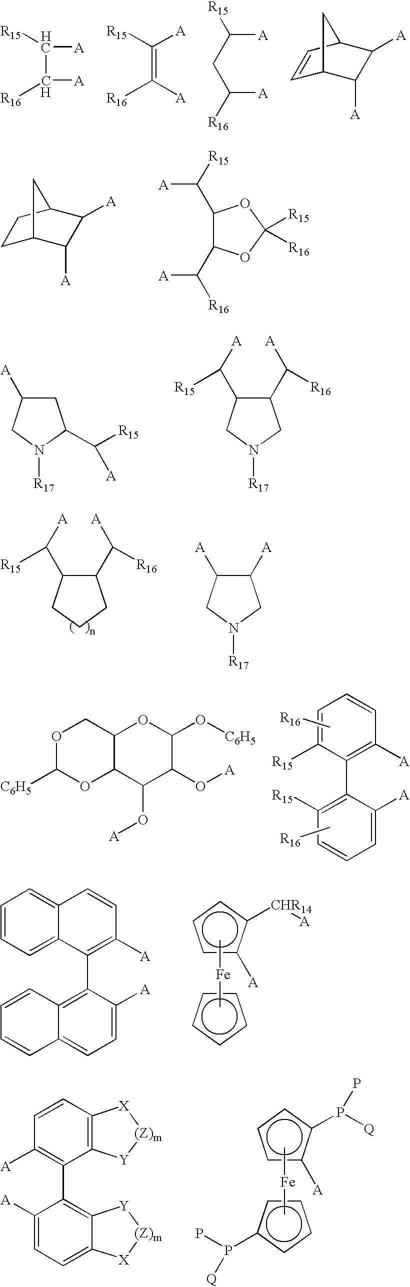 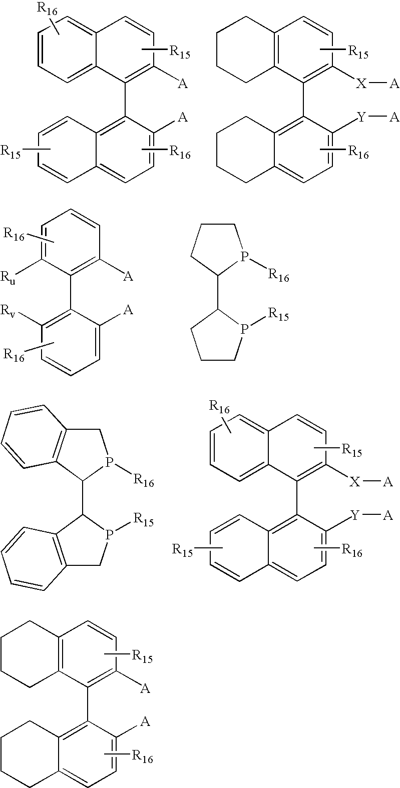
wherein

X, Y independently are CH₂, O, NR₁₇,

Z denotes CH₂, CF₂,

m independently for each Z is 1 or 2,

P, Q independently are the radicals of claim 18 or together form a ring having from 3 to 8 ring carbon atoms, being optionally heterocyclic having from 3 to 8 ring atoms and 1 or 2 hetero atoms from the group O, S and NR₁₅,

Rᵤ, Rᵥ are independently from each other R₁₅ or together form a ring having from 3 to 8 ring carbon atoms, being optionally heterocyclic having from 3 to 8 ring atoms and 1 or 2 hetero atoms from the group O, S and NR₁₅,

R₁₅ and R₁₆ are each independently of the other hydrogen, C₁-C₆alkyl, C₁-C₆alkoxy, halogen phenyl, benzyl, Si(R₁₄)₃, COOR₁₄, CN, C₁-C₆alkyn, or phenyl or benzyl having from 1 to 3 C₁-C₄alkyl or C₁-C₄alkoxy substituents, or R₁₅ and R₁₆ together can build a ring,

R₁₄ is hydrogen, C₁-C₄alkyl, phenyl, benzyl, or phenyl or benzyl having from 1 to 3 C₁-C₄alkyl or C₁-C₄alkoxy substituents,

R₁₇ is hydrogen, C₁-C₄alkyl, phenyl, benzyl, C₁-C₆alkoxy-CO—, C₁-C₆alkyl-CO—, phenyl-CO—, naphthyl-CO— or CᵣC₄alkylNH—CO—,

A may be identical or different groups —PR₂, wherein R is C₁-C₆alkyl, C₁-C₆alkoxy, cyclohexyl, phenyl, benzyl, or both R radicals may form a 4-8 member ring, or phenyl or benzyl having from 1 to 3 C₁-C₄alkyl, C₁-C₄alkoxy, —CF₃ or partially or fully fluorinated C₁-C₄alkoxy substituents, and

n is 0, 1 or 2.

Of those diphosphines, chirally substituted compounds are especially preferred.

Some preferred examples of diphosphines Y are as follows (Ph is phenyl):

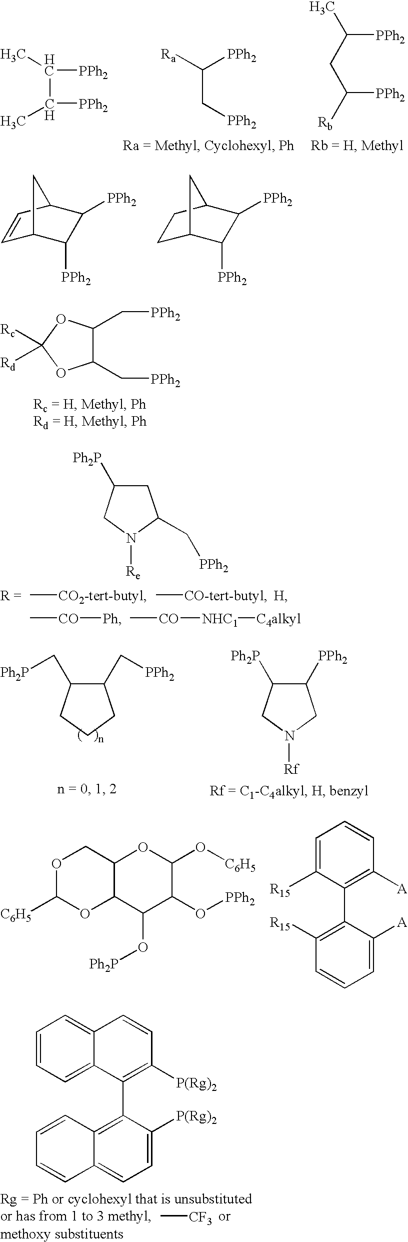

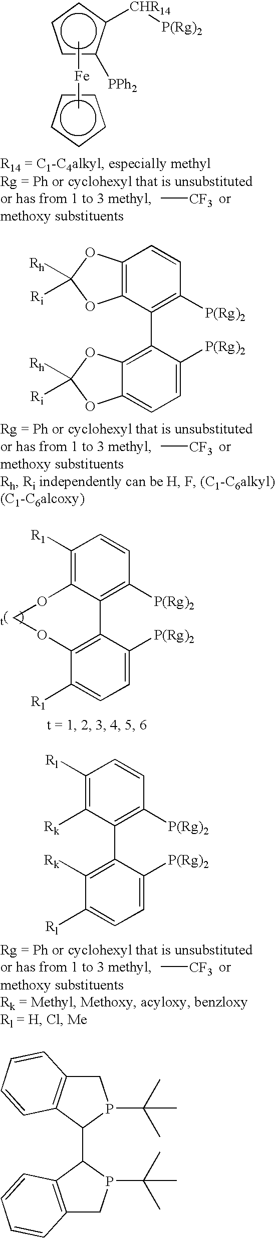

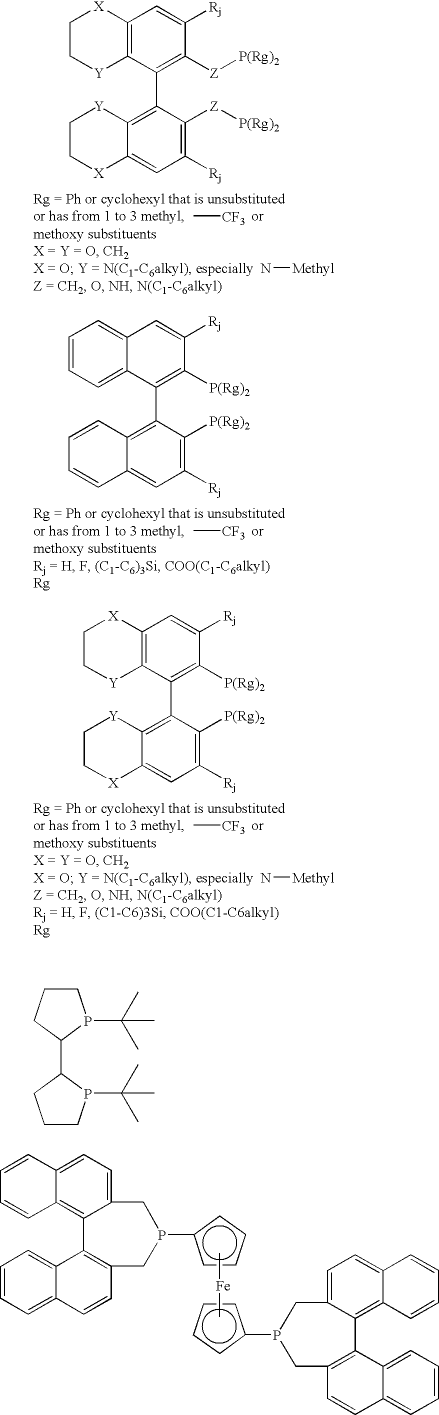

Suitable diphosphines and diphosphinites have been described, for example, by H. B. Kagan in Chiral Ligands for Asymmetrie Catalysis, Asymmetrie Synthesis, Volume 5, pp. 13-23, Academic Press, Inc., N.Y. (1985). The preparation of ferrocenyl diphosphine ligands is described, for example, in EP-A-0 564 406 and by T. Hayashi et al. in Bull. Chem. Soc. Jpn., 53, pages 1136-1151.

A⁻ in formula IIIa can be derived from inorganic or organic oxy acids. Examples of such acids are H₂SO₄, HClO₄, HClO₃, HBrO₄, HIO₄, HNO₃, H₃PO₃, H₃PO₄, CF₃SO₃H , C₆H₅SO₃H, CF₃COOH and CCl₃COOH. Complex acids from which A⁻ can be derived are, for example, the halo complex acids of the elements B, P, As, Sb and Bi. Preferred examples of A⁻ in formula IIIa are ClO₄⁻, CF₃SO₃⁻, BF₄⁻, B(phenyl)₄⁻, PF₆⁻, SbCl₆⁻, AsF₆⁻ and SbF₆⁻.

When M⁺ in formula IIIb is a phosphonium cation, it may be, for example
R_{w}RₓR_{y}R_{z}P⁺ (X)
wherein R_{w}, Rₓ, R_{y}, R_{z} independently can be H, halogen, linear or branched C₁-C₄₀alkyl, C₅-C₁₂-cycloalkyl , substituted or unsubstituted C₆-C₁₂aryl, substituted or unsubstituted C₄-C₁₁heteroaryl. Two of R_{w}, Rₓ, R_{y}, R_{z} can build a ring. R_{w}, Rₓ, R_{y}, R_{z} can also contain a polycyclic structure, like for example Adamantyl substituents. R_{w}, Rₓ, R_{y}, R_{z} independently from each one can contain at least one chiral center or they can be different and the chirality resides in the phosphorous atom, which can then be used as single enantiomer or as a mixture of enantiomers. Phosphonium halides can be prepared in the reaction of a primary, secondary or tertiary phosphine with an alkyl halogenated compound.

Z in formula III is preferably Br or Cl and especially Cl. Z in formula IIIb is preferably Br or I and Z in formula IIIc and IIId is preferably I.

Especially suitable diphosphine ligands which can preferably be used in catalysts of formula (III) are, for example:

{(R)-1-[(S)-2-diphenylphosphino)ferrocenyl]}ethyl-di(3,5-dimethyl-4-N,N-dipropyl-aminophenyl)phosphine

{(R)-1-[(S)-2-diphenylphosphino)ferrocenyl]}ethyl-di(3,5-diisopropyl-4-N,N-dimethyl-aminophenyl)phosphine

{(R)4-[(S)-2-diphenylphosphino)ferrocenyl]}ethyl-di(3,5-diisopropyl-4-N,N-dibenzylyl-aminophenyl)phosphine

{(R)-1-[(S)-2-diphenylphosphino)ferrocenyl]}ethyl-di(3,5-dimethyl-4-N,N-dibenzylyl-aminophenyl)phosphine

{(R)-1-[(S)-2-diphenylphosphino)ferrocenyl]}ethyl-di(3,5-dimethyl-4-(1′-pyrrolo)-phenyl)phosphine

{(R)-1-[(S)-2-diphenylphosphino)ferrocenyl]}ethyl-di(3,5-dimethyl-4-N,N-dipentyl-aminophenyl)phosphine

{(R)4-[(S)-2-diphenylphosphino)ferrocenyl]}ethyl-di(3,5-dimethyl-4-N,N-dimethyl-aminophenyl)phosphine

1,4-bis(diphenylphosphino)butane

{(R)-1-[(S)-2-di(4-methoxyphenyl)phosphino)ferrocenyl]}ethyl-di(3,5-dimethyl-4-N,N-dimethylaminophenyl)phosphine and preferably

{(R)-1-[(S)-2-diphenylphosphino)ferrocenyl]}ethyl-di(3,5-dimethyl-phenyl)phosphine.

The process according to the invention comprises the additional concomitant use of a phosphonium chloride, bromide or iodide. The phosphonium chlorides, bromides and iodides are used preferably in amounts of from 0.01 to 200 mol %, especially from 0.05 to 100 mol % and more especially from 0.5 to 50 mol %, based on the iridium catalyst. The iodides are preferred. Phosphonium is preferably trialkyl phosphonium halides having from 1 to 40 carbon atoms in the alkyl groups. Special preference is given to diadamantylbutylphosphonium iodide or diadamantylbenzylphosphonium bromide or triphenylisopropylphosphonium iodide or triphenylmethylphosphonium bromide. Other preferred phosphonium salts are triphenylmethylphosphonium bromide, diphenyl isopropyl phosphonium iodide, and triphenyl isopropyl phosphonium iodide. Reference is also made to formula (X) and its structural embodiments in this regard.

The reaction can be carried out in the absence or in the presence of solvents. Suitable solvents, which can be used alone or as a mixture of solvents, are especially aprotic solvents. Examples are:

aliphatic and aromatic hydrocarbons, such as pentane, hexane, cyclohexane, methylcyclohexane, benzene, toluene and xylene; ethers, such as diethyl ether, diethylene glycol dimethyl ether, tetrahydrofuran and dioxane; halogenated hydrocarbons, such as methylene chloride, chloroform, 1,1,2,2-tetrachloroethane and chlorobenzene; esters and lactones, such as ethyl acetate, butyrolactone and valerolactone; acid amides and lactams, such as dimethylformamide, dimethylacetamide and N-methylpyrrolidone, and ketones, such as acetone, dibutyl ketone, methyl isobutyl ketone and methoxyacetone.

The process according to the invention can be performed without adding an acid. However, it further embraces optionally the additional concomitant use of an acid. It may be an inorganic or, preferably, an organic acid. The acid is preferably used in at least the same molar amount as the iridium catalyst (equivalent to catalytic amounts) and can also be used in excess. The excess may even consist in the use of the acid as solvent. Preferably the acid is used from 0.001 to 50%, in particular from 0.1 to 50% by weight, based on the substrate to be hydrogenated. In many cases it can be advantageous to use anhydrous acids.

Some examples of inorganic acids are H₂SO₄, highly concentrated sulfuric acid (oleum), H₃PO₄, orthophosphoric acid, HF, HCl, HBr, HF HClO₄, HBF₄, HPF₆, HAsF₆, HSbCl₆, HSbF₆ and HB(phenyl)₄. H₂SO₄ is particularly preferred.

Examples of organic acids are aliphatic or aromatic, optionally halogenated (fluorinated or chlorinated) carboxylic acids, sulfonic acids, phosphorus(V) acids (for example phosphonic acids, phosphonous acids) having preferably from 1 to 20, especially from 1 to 12 and more especially from 1 to 6, carbon atoms. The organic acid can also contain at least one chiral center, like tartaric acid or camphorsulfonic acid. Other examples of organic acids are formic acid, acetic acid, propionic acid, butyric acid, benzoic acid, phenylacetic acid, cyclohexanecarboxylic acid, chloro- or fluoro-acetic acid, dichloro- or difluoro-acetic acid, trichloro- or trifluoro-acetic acid, chlorobenzoic acid, methanesulfonic acid, ethanesulfonic acid, benzenesulfonic acid, p-toluenesulfonic acid, chlorobenzenesulfonic acid, trifluoromethanesulfonic acid, methyl-phosphonic acid and phenylphosphonic acid. Preferred acids are acetic acid, propionic acid, trifluoroacetic acid, methanesulfonic acid and chloroacetic acid.

It is also possible for acidic ion exchangers of an inorganic or organic nature to be used as the acids, or metal oxides in gel form, like for example SiO₂, GeO₂, B₂O₃, Al₂O₃, TiO₂, ZrO₂ and combinations thereof Ion exchangers are known to the person skilled in the art and are described, for example, in Ullmaun's Enzyklopädie der Chemischen Technik, Volume 13, 4^{th} Edition, pages 281 to 284. Or the acids may be heteropolyacids which preferably consist of the elements Mo, V, W, O and H and also B, Si or P and secondary or trace elements. Such heteropolyacids are known and are described, for example, in Chemtech, page 23ff (November 1993) or Russian Chemicals Reviews, page 811ff (1987).

The preparation of the catalysts is known per se and is described, for example, in U.S. Pat. No. 4,994,615, U.S. Pat. No. 5,011,995, U.S. Pat. No. 5,112,999 and EP-A-0 564 406. The preparation of the catalysts of formula III can be carried out, for example, by reacting a diiridium complex of the formula [IrXZ]₂ with a diphosphine Y. The iridium catalysts can be added to the reaction mixture as isolated compounds. It has proved advantageous, however, to produce the catalyst in situ with or without a solvent prior to the reaction and to add optionally a phosphonium halide and eventually a portion or all of the acid.

The iridium catalysts are preferably used in amounts of from 0.0001 to 10 mol %, especially from 0.001 to 10 mol %, and more especially from 0.01 to 5 mol %, based on the imine.

The molar ratio of the imine to the iridium catalyst may be, for example, from 5 000 000 to 10, especially from 2 000 000 to 20, more preferably from 1000 000 to 20, and more especially from 500 000 to 100.

The process is carried out preferably at a temperature of from −20 to 100° C., especially from 0 to 80° C. and more especially from 10 to 70° C., and preferably at a hydrogen pressure of 2×10⁵ to 1.5×10⁷ Pa (5 to 150 bar), especially 10⁶ to 10⁷ Pa (10 to 100 bar).

The chlorides, bromides and iodides employed are preferably used in concentrations of from 0.01 to 500 mmol/l, especially from 0.01 to 50 mmol/l, based on the volume of the reaction mixture.

In detail, the process according to the invention can be carried out by first preparing the catalyst by dissolving, for example, (Ir-dieneCl)₂ in a solvent or an acid or both, adding a diphosphine and then a phosphonium halide and stirring the mixture. (Ir-dieneCl)₂ can also be used in solid form. A solution of imines is added to that catalyst solution (or vice versa) and, in an autoclave, hydrogen pressure is applied, thus removing the protective gas that is advantageously used. It is advantageous to ensure that the catalyst solution stands for only a short time, and to carry out the hydrogenation of the imines as soon as possible after the preparation of the catalyst. The reaction mixture is heated, if desired, and then hydrogenated. Where appropriate, when the reaction has ceased the reaction mixture is cooled and the autoclave is depressurised. The reaction mixture can be removed from the autoclave under pressure with nitrogen and the hydrogenated organic compound can be isolated and purified in a manner known per se, for example by precipitation, extraction or distillation.

In the case of the hydrogenation of aldimines and ketimines, the aldimines and ketmunies can also be formed in situ before or during the hydrogenation. In a preferred form, an amine and an aldehyde or a ketone are mixed together and added to the catalyst solution and the aldimnine or ketimine formed in situ is hydrogenated. It is also possible, however, to use an amine, a ketone or an aldehyde together with the catalyst as the initial batch and to add the ketone or the aldehyde or the amine thereto, either all at once or in metered amounts.

The hydrogenation can be carried out continuously or batchwise in various types of reactor. Preference is given to those reactors which allow comparatively good intermixing and good removal of heat, such as for example, loop reactors. That type of reactor has proved to be especially satisfactory when small amounts of catalyst are used.

The process according to the invention yields the corresponding arnines in short reaction times while having chemically a high degree of conversion, with surprisingly good optical yields (ee) of 30% or more being obtained even at relatively high temperatures of more than 50° C.

The hydrogenated organic compounds that can be prepared in accordance with the invention, for example the amines, are biologically active substances or are intermediates for the preparation of such substances, especially in the field of the preparation of pharmaceuticals and agrochemicals. For example, o,o-ialkylarylketamine derivatives, especially those having alkyl and/or alkoxyalkyl groups, are effective as fingicides, especially as herbicides. The derivatives may be amine salts, acid amides, for example of chloroacetic acid, tertiary amines and ammonium salts (see, for example, EP-A-0 077 755 and EP-A-0115470).

Especially important in this connection are the optically active amines of formula 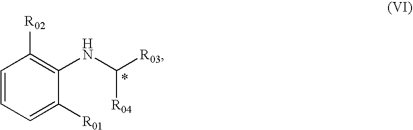
which can be prepared from the imines of formula (V) using the processes according to the invention, wherein R₀₁, R₀₂ and R₀₃ are each independently of the others C₁-C₄alkyl, and R₀₄ is C₁-C₄alkyl or C₁-C₄alkoxymethyl or C₁-C₄alkoxyethyl, and especially the amines of the formulae 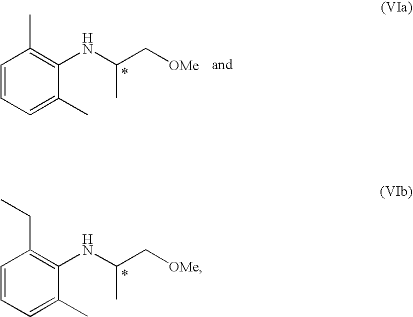
which can be prepared from the imines of the formula (Va) and (Vb) and which can be converted in accordance with methods that are customary per se with chloroacetic acid into the desired herbicides of the chloroacetanilide type.

The Examples that follow illustrate the invention in more detail but they are not intended to represent the whole number of possible hydrogenations. The chemical conversion is determined by gas chromatography [Optima-5-Amin; const. Flow (2.3 ml/min); 170° C. hold for 5 Min, then 2.5° C./min till 185° C.] or by High Performance Liquid Chromatography (HPLC) [chiralcel OJ 99 (Hexane)/1 (Hexane/iPrOH 9:1 containing 0.5% diethylamine at a flow of 0.3 ml/min and at 19° C.]. The optical yields (enantiomeric excess, ee) are determined by HPLC. The absolute stereochemistry has not been assigned.

The following trivial names will be used through the description of the examples:

Xyliphos:

{(R)-1-[(S)-2-diphenylphosphino)ferrocenyl]}ethyl-di(3,5-di-ethylphenyl)phosphine

(R,R)-Chiraphos: (2R,3R)-(−)Bis(diphenylphosphino)butan

CataCXium A-HI: Diadamantyl butyl phosphonium hydroiodide

CataCXium ABn-HI: Diadamantyl benzyl phosphonium hydrobromide

AcOH: Acetic acid

Bu₄NI: Tetrabutyl ammonium iodide

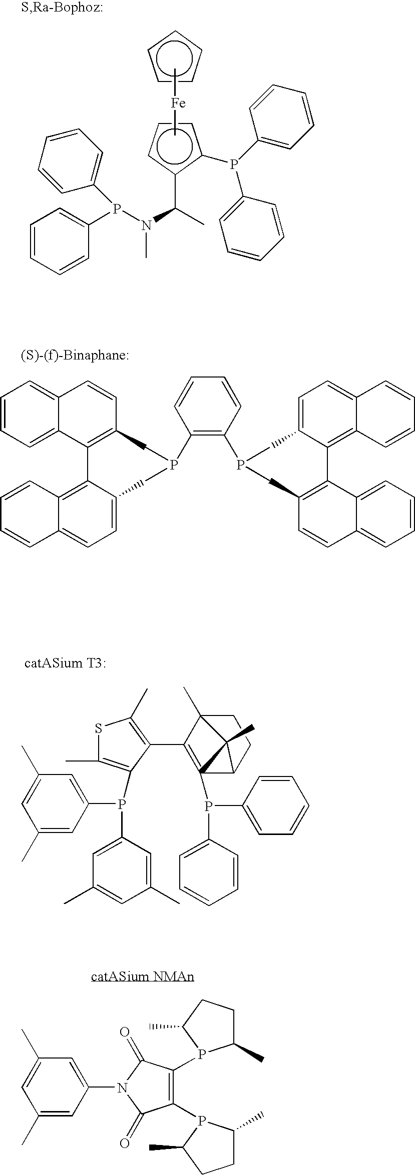

### EXAMPLES

### Example 1

The following example illustrates the synthesis of some of the phosphonium halides (see Beller et al. in Synthesis 2004, 934-941)

Synthesis of Diadamanthyl benzyl phosphonium bromide

Diadamanthyl phosphine (10 mmol, 3.02 g) were suspended in the air in 6 ml benzylbromide and 40 ml dibutylether. The reaction mixture was stirred at 138° C. for 16 h. After cooling down, the solids were filtrated, washed with MTBE and dried. Yield: 4.05 g, 86% (white powder)

### Example 2

Hydrogenation of 2,4,6-Trimethyl-N-(4-methylpentan-2-ylidene)aniline using different phosphonium halides and ligands (for comparison effects some experiments using Bu₄NI are also given):

The desired additive (0.004-0.006 mmol) was weighted in a 1.5ml GC-flask under argon (Glovebox). Then, 300 μl of toluene was added. Subsequently, 50 μl of a 0.01M solution of [Ir(1,5-cyclooctadiene)Cl]₂ in toluene (0.0005 mmol) and 50 μl of a 0.02M solution of a ligand in toluene (0.001 mmol) were added. The mixture was stirred for 15 minutes at room temperature. Then, 250 μl of neat substrate or 200 μl of a 2 M or 1M solution of substrate in toluene were added. The flask was closed with a septum which was pierced several times with a needle and placed in an aluminium microtiterplate and introduced in an autoclave. The autoclave was purged with hydrogen and 55 bar hydrogen were introduced. The temperature was set to 65° C. and the stirring was started for 1.75 h. The pressure was released and after cooling down 50 μl of each reaction mixture were evaporated, the residues dissolved in 200 μl isopropanol and 1 ml hexane and filtered through a short path of silica gel and the reactions were analysed by GC or HPLC.

| | | | | Conversion | ee |
|---|---|---|---|---|---|
| Ligand | S/C | Halide | Acid | (%) | (%) |
| Xyliphos | 400 | none | none | 6 | not |
| | | | | | determined |
| Xyliphos | 400 | Bu₄NI | none | 8 | not |
| | | | | | determined |
| Xyliphos | 400 | cataCXium A-HI | none | 88 | 39 |
| Xyliphos | 1000 | none | none | 0 | — |
| Xyliphos | 1000 | Bu₄NI | none | 0 | — |
| Xyliphos | 1000 | cataCXium A-HI | none | 77 | 39 |

### Example 3

The following examples were done in a similar way as Example 2 but changing the ligand, solvent, the reaction time, reaction temperature and/or the phosphonium halide:

| | | | Reaction | | | | | |
|---|---|---|---|---|---|---|---|---|
| | | | time | T | P | | Conversion | ee |
| Ligand | S/C | Halide | (h) | (° C.) | (bar) | Solvent | (%) | (%) |
| R,R-Chiraphos | 200 | none | 1.75 | 65 | 55 | Toluene | 0 | 0 |
| R,R-Chiraphos | 200 | Bu₄NI | 1.75 | 65 | 55 | Toluene | 0 | 0 |
| R,R-Chiraphos | 200 | cataCXium A-HI | 1.75 | 65 | 55 | Toluene | 4 | 20 |
| Xyliphos | 200 | none | 1.75 | 65 | 55 | Toluene | 62 | 37 |
| Xyliphos | 200 | cataCXium A-HI | 1.75 | 65 | 55 | Toluene | 100 | 39 |
| Xyliphos | 200 | Ph₃P(ⁱPr)I | 1.75 | 65 | 55 | Toluene | 70 | 39 |
| Xyliphos | 200 | cataCXium ABn-HBr | 1.75 | 65 | 55 | Toluene | 100 | 43 |
| Xyliphos | 200 | Ph₃PMeBr | 3.5 | 65 | 55 | Toluene | 90 | 38 |
| S,Ra-Bophoz | 200 | none | 1.75 | 65 | 55 | Toluene | 23 | 16 |
| S,Ra-Bophoz | 200 | Ph₃P(ⁱPr)I | 1.75 | 65 | 55 | Toluene | 35 | 15 |
| S,Ra-Bophoz | 200 | cataCXium ABn-HBr | 1.75 | 65 | 55 | Toluene | 60 | 0 |
| S,Ra-Bophoz | 200 | Ph₃PMeBr | 3.5 | 65 | 55 | Toluene | 60 | 10 |
| catASium T3 | 200 | none | 1.75 | 65 | 55 | Toluene | 0 | 0 |
| catASium T3 | 200 | cataCXium ABn-HBr | 1.75 | 65 | 55 | Toluene | 19 | 28 |
| catASium MNAn | 200 | Ph₃PMeBr | 3.5 | 65 | 55 | Toluene | 5 | 8 |
| (S)-(f)-Binaphane | 200 | Ph₃PMeBr | 3.5 | 65 | 55 | Toluene | 75 | 19 |
| Xyliphos | 2000 | none | 4.5 | 50 | 64 | CH₂Cl₂ | 0 | 0 |
| Xyliphos | 2000 | cataCXium A-HI | 4.5 | 50 | 64 | CH₂Cl₂ | 39 | 38 |
| Xyliphos | 2000 | none | 4.5 | 50 | 64 | 1,4-Dioxane | 3 | 58 |
| Xyliphos | 2000 | cataCXium A-HI | 4.5 | 50 | 64 | 1,4-Dioxane | 86 | 44 |
| Xyliphos | 2000 | none | 4.5 | 50 | 64 | none | 0 | 0 |
| Xyliphos | 2000 | cataCXium A-HI | 4.5 | 50 | 64 | none | 72 | 40 |
| Xyliphos | 2000 | Bu₄NI | 4.5 | 50 | 64 | none | 4 | nd |
| Xyliphos | 2000 | cataCXium ABn-HBr | 4.5 | 50 | 64 | none | 37 | 42 |

### Example 4

Hydrogenation of 2,4,6-Trimethyl-N-(4-methylpentan-2-ylidene)aniline using different halides and ligands in the presence of an additional acid (for comparison effects some experiments using Bu₄NI are also given):

In a 1.5 ml GC-flask under argon (Glovebox) the desired additive (0.004-0.006 mmol) was weighted. Then, 300 μl of acetic acid was added. Subsequently, 50 μl of a 0.01M solution of [Ir(1,5-cyclooctadiene)Cl]₂ in toluene (0.0005 mmol) and 50 μl of a 0.02M solution of a ligand in toluene (0.001 mmol) were added. The mixture was stired for 15 minutes at room temperature. Then, 250 μl of neat substrate or 200 μl of a 2 M or 1M solution of substrate in toluene were added. The flask was closed with a septum which was pierced several times with a needle and placed in an aluminium microtiterplate and introduced in an autoclave. The autoclave was purged with hydrogen and 55 bar hydrogen were introduced. The temperature was set to 65° C. and the stirring was started for 1.75 h. The pressure was released and after cooling down 50 μl of each reaction mixture were evaporated, the residues dissolved in 200 μl isopropanol and 1 ml hexane and filtered through a short path of silica gel and the reactions were analysed by GC or HPLC.

| | | | | Conversion | ee |
|---|---|---|---|---|---|
| Ligand | S/C | Halide | Acid | (%) | (%) |
| Xyliphos | 400 | Bu₄NI | AcOH | 35 | 32 |
| Xyliphos | 400 | cataCXium A-HI | AcOH | 37 | 32 |
| Xyliphos | 1000 | Bu₄NI | AcOH | 41 | 31 |
| Xyliphos | 1000 | cataCXium A-HI | AcOH | 46 | 31 |

### Example 5

The following examples were done in a similar way as Example 4 but changing the ligand, solvent, the reaction time, reaction temperature and/or the phosphonium halide and acid. Except otherwise indicated, acetic acid was used:

| | | | Reaction | | | | | |
|---|---|---|---|---|---|---|---|---|
| | | | time | T | P | | Conversion | ee |
| Ligand | S/C | Halide | (h) | (° C.) | (bar) | Solvent | (%) | (%) |
| Xyliphos | 200(*) | cataCXium A-HI | 1.75 | 65 | 55 | Toluene | 100 | 39 |
| Xyliphos | 200 | Ph₃P(ⁱPr)I | 1.75 | 65 | 55 | Toluene | 50 | 29 |
| Xyliphos | 200 | cataCXium ABn-HBr | 1.75 | 65 | 55 | Toluene | 50 | 29 |
| Xyliphos | 200 | Ph₃PMeBr | 3.5 | 65 | 55 | Toluene | 61 | 27 |
| S,Ra-Bophoz | 200 | Ph₃P(ⁱPr)I | 1.75 | 65 | 55 | Toluene | 2 | 4 |
| S,Ra-Bophoz | 200 | cataCXium ABn-HBr | 1.75 | 65 | 55 | Toluene | 0 | 0 |
| S,Ra-Bophoz | 200 | Ph₃PMeBr | 3.5 | 65 | 55 | Toluene | 29 | 16 |
| catASium MNAn | 200 | Ph₃PMeBr | 3.5 | 65 | 55 | Toluene | 30 | 0 |
| Xyliphos | 2000 | none | 4.5 | 50 | 64 | CH₂Cl₂ | 0 | 0 |
| Xyliphos | 2000 | cataCXium A-HI | 4.5 | 50 | 64 | CH₂Cl₂ | 37 | 31 |
| Xyliphos | 2000 | none | 4.5 | 50 | 64 | 1,4-Dioxane | 2 | 0 |
| Xyliphos | 2000 | cataCXium A-HI | 4.5 | 50 | 64 | 1,4-Dioxane | 55 | 38 |
| Xyliphos | 2000 | none | 4.5 | 50 | 64 | none | 3 | Nd |
| Xyliphos | 2000 | cataCXium A-HI | 4.5 | 50 | 64 | none | 48 | 32 |
| Xyliphos | 2000 | Bu₄NI | 4.5 | 50 | 64 | none | 56 | 31 |
| Xyliphos | 2000 | cataCXium ABn-HBr | 4.5 | 50 | 64 | none | 32 | 31 |

(*)In this case SiO₂ impregnated in H₂SO₄ 4M was used as acid.

### Example 6

In situ preparation of Preparation of 2,4,6-Trimethyl-N-(4-methylpentan-2-ylidene)aniline and its hydrogenation.

In a 4 ml GC-flask under argon (Glovebox) the desired additive (0.004-0.006 mmol) was weighted. Then, 1000 μl of toluene or 1000 μl of acetic acid was added. Then, 50 μl of a 0.01M solution of [Ir(1,5-cyclooctadiene)Cl]₂ in toluene (0.0005 mmol) and 50 μl of a 0.02M solution of a ligand in toluene (0.001 mmol) were added. The mixture was stirred for 15 minutes at room temperature. Then, 437 mg of neat substrate was added. The flask was closed with a septum which was pierced several times with a needle and the flask was introduced in an autoclave. The autoclave was purged with hydrogen and 60 bar of hydrogen were pressed. The reaction mixtures were stirred at 65° C. for 18 h.

| | | | | Conversion | ee |
|---|---|---|---|---|---|
| Ligand | S/C | Halide | Acid | (%) | (%) |
| Xyliphos | 2000 | cataCXium A-HI | none | 53 | 32 |
| Xyliphos | 2000 | cataCXium A-HI | AcOH | 8 | 26 |
| Xyliphos | 2000 | Bu₄NI | AcOH | 9 | 27 |

Obviously, numerous modifications and variations on the present invention are possible in light of the above teachings. It is, therefore, to be understood that within the scope of the accompanying claims, the invention may be practiced otherwise than as specifically described herein.

1. A process for the hydrogenation of an imine with hydrogen under elevated pressure in the presence of an iridium catalyst and with or without an inert solvent, wherein the reaction mixture comprises a phosphonium chloride, bromide or iodide and optionally contains an acid.

2. A process according to claim 1, wherein the imine contains at least one 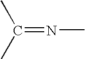
group.

3. A process according to claim 1, wherein the imine contains at least one of the groups 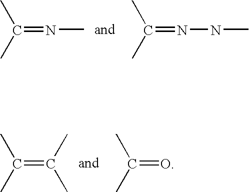
and additionally unsaturated groups

4. A process according to claim 3, wherein the free bonds are saturated with hydrogen or organic radicals having from 1 to 22 carbon atoms or organic hetero radicals having from 1 to 20 carbon atoms, and at least one hetero atom from the group O, S, N and P; or the nitrogen atom of the group 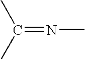
is saturated with NH₂ or a primary amino group having from 1 to 22 carbon atoms or a secondary amino group having from 2 to 40 carbon atoms.

5. A process according to claim 1, wherein an aldimine, ketimine oxime or hydrazone is hydrogenated.

6. A process according to claim 5, wherein the imine is an irnine of formula I 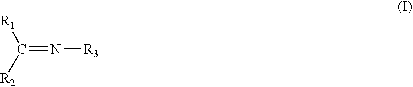
which is hydrogenated to form an amine of formula II 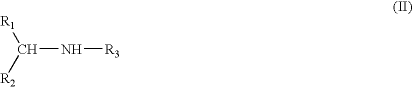
wherein
R₃ is linear or branched C₁-C₁₂alkyl, cycloalkyl having from 3 to 8 ring carbon atoms; heterocycloalkyl bonded via a carbon atom and having from 3 to 8 ring atoms and 1 or 2 hetero atoms from the group O, S and NR₆ a C₇-C₁₆aralkyl bonded via an alkyl carbon atom or C₁-C₁₂alkyl substituted by the mentioned cycloalkyl or heterocycloalkyl or heteroaryl;
or wherein
R₃ is C₆-C₁₂aryl, or C₄-C₁₁heteroaryl bonded via a ring carbon atom and having 1 or 2 hetero atoms in the ring; R₃ being unsubstituted or substituted by —CN, —NO₂, F, Cl, C₁-C₁₂alkyl, C₁-C₁₂alkoxy, C₁-C₁₂alkylthio, C₁-C₆haloalkyl, —OH, C₆-C₁₂-aryl or -aryloxy or -arylthio, C₇-C₁₆-aralkyl 7-C₁₆-aralkyl or -aralkoxy or -aralkylthio, secondary amino having from 2 to 24 carbon atoms, —CONR₄R₅ or by —COOR₄, and the aryl radicals and the aryl groups in the aralkyl, aralkoxy and aralkylthio in turn being unsubstituted or substituted by —CN, —NO₂, F, Cl, C₁-C₄-alkyl, -alkoxy or -alkylthio, —OH, —CONR₄R₅ or by —COOR₄,
R₄ and R₅ are each independently of the other hydrogen, C₁-C₁₂alkyl, phenyl or benzyl, or
R₄ and R₅ together are tetra- or penta-methylene or 3-oxapentylene;
R₆ has independently the same meaning as given for R₄;
R₁ and R₂ are each independently of the other a hydrogen atom, C₁-C₁₂alkyl or cycloalkyl having from 3 to 8 ring carbon atoms, each of which is unsubstituted or substituted by —OH, C₁-C₁₂alkoxy, phenoxy, benzyloxy, secondary amino having from 2 to 24 carbon atoms, —CONR₄R₅ or by —COOR₄; C₆-C₁₂aryl or C₇-C₁₆aralkyl that is unsubstituted or substituted as R₃, or —CONR₄R₅ or —COOR_{4,} wherein R₄ and R₅ are as defined
hereinbefore; or
R₃ is as defined hereinbefore and R₁ and R₂ together are alkylene having from 2 to 5 carbon atoms that is optionally interrupted by 1 or 2 —O—, —S— or —NR₆— radicals, and/or unsubstituted or substituted by ═O or as R₁ and R₂ above in the meaning of alkyl, and/or condensed with benzene, pyridine, pyrimidine, firan, thiophene or pyrrole; or
R₂ is as defined hereinbefore and R₁ and R₃ together are alkylene having from 2 to 5 carbon atoms that is optionally interrupted by 1 or 2 —O—, —S— or —NR₆— radicals, and/or unsubstituted or substituted by ═O or as R₁ and R₂ above in the meaning of alkyl, and/or condensed with benzene, pyridine, pyrimidine, furan, thiophene or pyrrole.

7. A process according to claim 6, wherein R₁, R₂ or R₃ as heteroaryl form a 5- or 6-membered ring having 1 or 2 identical or different hetero atoms.

8. A process according to claim 6, wherein R₁, R₂ or R₃ as heteroaryl-substituted alkyl are derived from a 5- or 6-membered ring having 1 or 2 identical or different hetero atoms.

9. A process according to claim 6, wherein R₁, R₂ or R₃ as heterocycloalkyl or as heterocycloalkyl-substituted alkyl contain from 4 to 6 ring atoms and 1 or 2 identical or different hetero atoms from the group O, S and NR₆, wherein R₆ is hydrogen, C₁-C₁₂alkyl, phenyl or benzyl.

10. A process according to claim 6, wherein R₁, R₂ or R₃ as alkyl are unsubstituted or substituted C₁-C₆alkyl.

11. A process according to claim 6, wherein R₁, R₂ or R₃ as unsubstituted or substituted cycloalkyl contain from 3 to 6 ring carbon atoms.

12. A process according to claim 6, wherein R₁, R₂ or R₃ as aryl are unsubstituted or substituted naphthyl or phenyl, and R₁, R₂ or R₃ as aralkyl are unsubstituted or substituted phenylalkyl having from 1 to 10 carbon atoms in the alkylene.

13. A process according to claim 6, wherein R₁ and R₂ together or R₁ and R₃ together form, with the carbon atom or the —N═C group to which they are bonded, respectively, a 5- or 6-membered ring.

14. A process according to claim 6, wherein in formula I R₃ is 2,6-di-C₁-C₄alkylphen-1-yl, R₁ is C₁-C₄alkyl, and R₂ is C₁-C₄alkyl, C₁-C₄alkoxymethyl or C₁-C₄alkoxyethyl.

15. A process according to claim 14, wherein R₃ is 2,6-dimethylphen-1-yl or 2-methyl-6-ethylphen-1-yl, R₁ is ethyl or methyl, and R₂ is methoxymethyl.

16. A process according to claim 6, wherein the imine corresponds to the formula

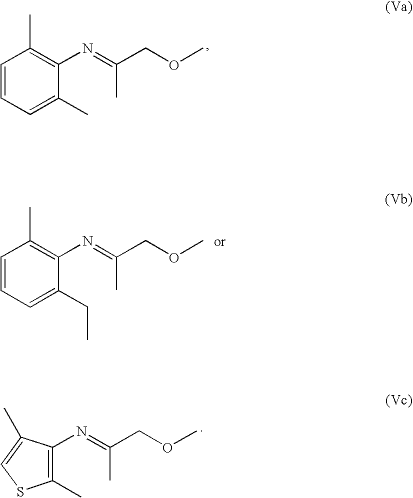

17. A process according to claim 1, wherein the iridium catalyst is a homogeneous catalyst that is substantially soluble in the reaction medium.

18. A process according to claim 1, wherein the catalyst corresponds to the formula III, IIIa, IIIb, IIIc or IIId
[XIrYZ] (III),
[XirY]⁺A⁻ (IIIa),
[YirZ₄]⁻M⁺ (IIIb),
[YirHZ₂]₂ (IIIc),
[YirZ₃]₂ (IIId),
wherein X is two olefin ligands or a diene ligand, Y is a ditertiary diphosphine
(a) the phosphine groups of which are bonded to different carbon atoms of a carbon chain having from 2 to 4 carbon atoms, or
(b) the phosphine groups of which are either bonded directly or via a bridge group —CRₐR_{b}— in the ortho positions of a cyclopentadienyl ring or are each bonded to a cyclopentadienyl ring of a ferrocenyl, or
(c) one phosphine group of which is bonded to a carbon chain having 2 or 3 carbon atoms and the other phosphine group of which is bonded to an oxygen atom or a nitrogen atom bonded terminally to that carbon chain, or
(d) the phosphine groups of which are bonded to the two oxygen atoms or nitrogen atoms bonded terminally to a C₂-carbon chain;
with the result that in the cases of (a), (b), (c) and (d) a 5-, 6- or 7-membered ring is formed together with the Ir atom, the radicals Z are each independently of the other(s) Cl, Br or I, A⁻ is the anion of an oxy or complex acid, and M⁺ is a phosphonium cation, and Rₐ and R_{b}, are each independently of the other hydrogen, C₁-C₈alkyl, C₁-C₄fluoroalkyl, phenyl or benzyl or are phenyl or benzyl having from 1 to 3 C₁-C₄alkyl or C₁-C₄alkoxy substituents.

19. A process according to claim 18, wherein the diphosphine Y contains at least one chiral carbon atom.

20. A process according to claim 18, wherein X as an olefin ligand is branched or linear C₂-C₁₂alkylene; and X as a diene ligand is an open-chain or cyclic diene having from 4 to 12 carbon atoms.

21. A process according to claim 18, wherein the secondary phosphine groups contain two identical or different radicals from the following group: linear or branched C₁-C₁₂alkyl; unsubstituted or C₁-C₆alkyl- or C₁-C₆alkoxy-substituted C₅-C₁₂cycloalkyl, C₅-C₁₂cycloalkyl-CH₂—, phenyl or benzyl; or phenyl or benzyl substituted by halogen (e.g. F, Cl or Br), C₁-C₆haloalkyl, (C₁-C₁₂alkyl)₃Si, (C₆H₅)₃Si, C₁-C₆haloalkoxy (e.g. trifluoromethoxy), —NH₂, phenyl₂N—, benzyl₂N—, morpholinyl, piperidinyl, pyrrolidinyl, (C₁-C₁₂alkyl)₂N—, -ammonium-X₁⁻, —SO₃M₁, —CO₂M₁, —PO₃M₁ or by —COO-C₁-C₆-alkyl (e.g. —COOCH₃), wherein M₁ is an alkali metal or hydrogen and X₁⁻ is the anion of a monobasic acid.

22. A process according to claim 18, wherein the diphosphine Y is of the formula: 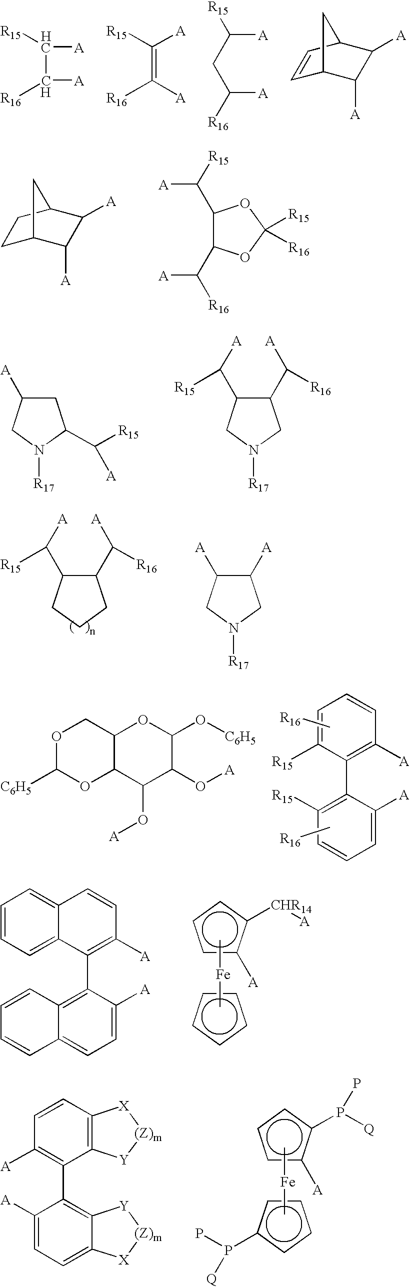 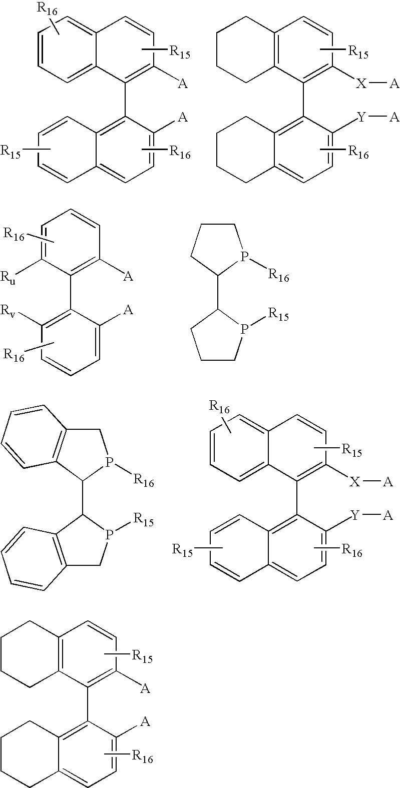
wherein
X, Y independently are CH₂, O, NR₁₇,
Z denotes CH₂, CF₂,
m independently for each Z is 1 or 2,
P, Q independently are the radicals of claim 18 or together form a ring having from 3 to 8 ring carbon atoms, being optionally heterocyclic having from 3 to 8 ring atoms and 1 or 2 hetero atoms from the group O, S and NR₁₅,
Rᵤ, Rᵥ are independently from each other R₁₅ or together form a ring having from 3 to 8 ring carbon atoms, being optionally heterocyclic having from 3 to 8 ring atoms and 1 or 2 hetero atoms from the group O, S and NR₁₅,
R₁₅ and R₁₆ are each independently of the other hydrogen, C₁-C₆alkyl, C₁-C₆alkoxy, halogen phenyl, benzyl, Si(R₁₄)₃, COOR₁₄, CN, C₁-C₆alkyn, or phenyl or benzyl having from 1 to 3 C₁-C₄alkyl or C₁-C₄alkoxy substituents, or R₁₅ and R₁₆ together can build a ring,
R₁₄ is hydrogen, C₁-C₄alkyl, phenyl, benzyl, or phenyl or benzyl having from 1 to 3 C₁-C₄alkyl or C₁-C₄alkoxy substituents,
R₁₇ is hydrogen, C₁-C₄alkyl, phenyl, benzyl, C₁-C₆alkoxy-CO—, C₁-C₆alkyl-CO—, phenyl-CO—, naphthyl-CO— or CᵣC₄alkylNH-CO—,
A may be identical or different groups —PR₂, wherein R is C₁-C₆alkyl, C₁-C₆alkoxy, cyclohexyl, phenyl, benzyl, or both R radicals may form a 4-8 member ring, or phenyl or benzyl having from 1 to 3 C₁-C₄alkyl, C₁-C₄alkoxy, —CF₃ or partially or fuilly fluorinated C₁-C₄alkoxy substituents, and
n is 0, 1 or 2.

23. A process according to claim 18, wherein the diphosphine Y is:
{(R)-1-[(S)-2-diphenylphosphino)ferrocenyl]}ethyl-di(3,5-dimethyl-4-N,N-dipropyl-aminophenyl)phosphine
{(R)-1-[(S)-2-diphenylphosphino)ferrocenyl]}ethyl-di(3,5-diisopropyl-4-N,N-dimethyl-aminophenyl)phosphine
{(R)4-[(S)-2-diphenylphosphino)ferrocenyl]}ethyl-di(3,5-diisopropyl-4-N,N-dibenzylyl-aminophenyl)phosphine
{(R)-1-[(S)-2-diphenylphosphino)ferrocenyl]}ethyl-di(3,5-dimethyl-4-N,N-dibenzylyl-aminophenyl)phosphine
{(R)-1-[(S)-2-diphenylphosphino)ferrocenyl]}ethyl-di(3,5-dimethyl-4-(1′-pyrrolo)-phenyl)phosphine
{(R)-1-[(S)-2-diphenylphosphino)ferrocenyl]}ethyl-di(3,5-dimethyl-4-N,N-dipentyl-aminophenyl)phosphine
{(R)4-[(S)-2-diphenylphosphino)ferrocenyl]}ethyl-di(3,5-dimethyl-4-N,N-dimethyl-aminophenyl)phosphine
1,4-bis(diphenylphosphino)butane
{(R)-1-[(S)-2-di(4-methoxyphenyl)phosphino)ferrocenyl]}ethyl-di(3 ,5-dimethyl-4-N,N-dimethylaminophenyl)phosphine and preferably
{(R)-1-[(S)-2-diphenylphosphino)ferrocenyl]}ethyl-di(3,5-dimethyl-phenyl)phosphine.

24. A process according to claim 1, wherein the phosphonium chloride, bromide or iodide is used in an amount of from 0.01 to 200 mol %, based on the Iridium catalyst.

25. A process according to claim 1, wherein the phosphonium chloride, bromide or iodide correspond to the formula:
R_{w}RₓR_{y}R_{z}P⁺ (X)
wherein
R_{w}, Rₓ, R_{y}, R_{z} independently can be H, halogen, linear or branched C₁-C₄₀alkyl, C₅-C₁₂-cycloalkyl, substituted or unsubstituted C₆-C₁₂aryl, substituted or unsubstituted C₄-C₁₁heteroaryl or two of R_{w}, Rₓ, R_{y}, R_{z} can build a cyclus or a polycyclic structure.

26. A process according to claim 25, wherein the phosphonium halide is diadamantylbutyl phosphonium iodide or diadamantylbenzylphosphonium bromide or triphenylisopropylphosphonium iodide or triphenylmethylphosphonium bromide.

27. A process according to claim 1, where no acid is added.

28. A process according to claim 1, where the acid is an inorganic or organic acid.

29. A process according to claim 1, wherein the acid is used in an amount of from 0.001 to 50% by weight, preferably 0.1 to 50% by weight, based on the imine.

30. A process according to claim 28, wherein the organic acid is an aliphatic or aromatic carboxylic acid, sulfonic acid or phosphorus(V) acid.

31. A process according to claim 28, wherein the organic acid is acetic acid, propionic acid, trifluoroacetic acid, chloroacetic acid or methanesulfonic acid, and the inorganic acid is H₂SO₄.

32. A process according to claim 1, wherein the molar ratio of the imine to the iridium catalyst is from 500 000 to 10.

33. A process according to claim 1, wherein the reaction temperature is from −20 to 100° C.

34. A process according to claim 1, wherein the hydrogen pressure is from 5 to 150 bar.

35. A process according to claim 1, wherein the hydrogenation is carried out in a loop reactor.

36. A process according to claim 1, wherein an aldimine or a ketimine formed in situ before or during the hydrogenation is hydrogenated.

37. A process for the preparation of a compound of the formula 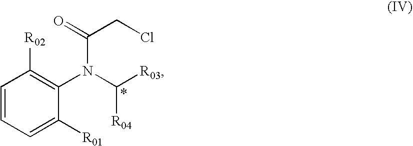
wherein R₀₁, R₀₂ and R₀₃ are each independently of the other C₁-C₄alkyl, and R₀₄ is C₁-C₄alkyl or C₁-C₄alkoxymethyl or C₁-C₄alkoxyethyl, by (1) hydrogenation of an imine of the formula 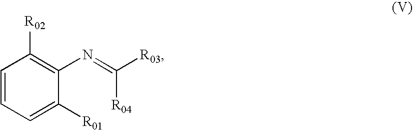
with hydrogen in the presence of an iridium catalyst and with or without an inert solvent to form an amine of the formula 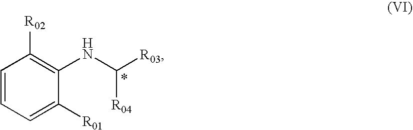
and (2) reaction thereof with the compound of formula
ClCH₂CO—Cl (VII),
wherein in the hydrogenation the reaction mixture contains a phosphonium chloride, bromide or iodide, and optionally contains an acid.

38. A process according to claim 37, wherein the imine used is a compound of the formula [chemical structure]
